(19) United States
(12) Patent Application Publication
(10) Pub. No.: US 2026/0363803 A1
(43) Pub. Date: Nov. 19, 2026

(54) CONTROL SYSTEM OF A SURGICAL ROBOT

(71) Applicant: CMR SURGICAL LIMITED, Cambridge, Cambridgeshire (GB)

(72) Inventors: Isabelle Ascah-Coallier, Cambridge (GB); Edward James Wildin Tucker, Cambridge (GB)

(21) Appl. No.: 18/995,999

(22) PCT Filed: Aug. 10, 2023
(86) PCT No.: PCT/GB2023/052115

§ 371 Date: Jan. 17, 2025

(30) Foreign Application Priority Data
Aug. 15, 2022 (GB) 2211898.8

(51) Int. Cl. A61B 34/37; A61B 34/00; A61B 34/30

(52) U.S. Cl. CPC A61B 34/37; A61B 34/77; A61B 2034/302

(57) ABSTRACT

A control system for a surgical robotic system, the surgical robotic system comprising a surgical robot arm and a surgeon input device, the surgical robot arm comprising a series of joints by which its configuration can be altered, the series of joints extending from a base at a proximal end of the surgical robot arm to an attachment for a surgical instrument at a distal end of the surgical robot arm, the control system being configured to control the surgical robot arm, in dependence on inputs received at the surgeon input device, to alter the configuration of the surgical robot arm whilst maintaining an intersection between a surgical instrument attached to the surgical robot arm and a fulcrum, in which the control system is configured to: receive an input from the surgeon input device, said input indicating a requested movement of the surgical instrument; determine a scaling factor using a scaling function that is dependent on the distance between a part of the surgical instrument and the fulcrum; generate a control signal for the surgical robot arm in dependence on the input from the surgeon input device and the determined scaling factor; and send the generated control signal to the surgical robot arm in order to drive the surgical robot arm such that its configuration is altered so as to cause movement of the surgical instrument.

### BACKGROUND

This invention relates to a control system for a surgical robotic system.

It is known to use robots for assisting and performing surgery. FIG. 1 illustrates an example surgical robotic system 100, which comprises a surgical robot arm 101 for manipulating tissue. The surgical robot arm 101 comprises a base 109. The base supports the surgical robot arm, and is itself attached rigidly to, for example, the operating theatre floor, the operating theatre ceiling or a trolley. The surgical robot arm 101 is articulated by means of multiple joints 104 along its length, which are used to locate a robotic surgical instrument 106 in a desired location relative to a patient 102. The robotic surgical instrument 106 could, for example, be a cutting or grasping device. A robotic surgical instrument 106 is attached to the distal end of the surgical robot arm 101. The robotic surgical instrument 106 is inserted into the body of the patient 102 (optionally, via an access port 117) so as to access a surgical site within the body of the patient 102. At its distal end the robotic surgical instrument comprises an end effector for performing aspects of a medical procedure. This type of medical procedure is often referred to as a minimally invasive surgical procedure.

The configuration of the surgical robot arm 101 may be remotely controlled in response to inputs received at a remote surgeon console 120. A surgeon may provide inputs to the remote console. The remote surgeon console may comprise one or more surgeon input devices 123. For example, these may take the form of one or more hand controllers, foot pedals, interactive touch screens etc. A video feed of the surgical site may be captured by an endoscope, often attached to a further surgical robot arm (not shown in FIG. 1 for simplicity), and displayed at a display 121 of the remote surgeon console.

A control system 124 connects the surgeon console 120 and the surgical robot arm 101. The control system 124 receives inputs from the surgeon input device(s) 123 and converts those inputs to control signals for controlling the surgical robot arm 101. It is desirable for the control system to control the surgical robot arm such that the surgical instrument 106 can access as much of the surgical site as possible, whilst also preventing the surgical instrument 106 from impinging on the access port 117 and avoiding kinematic singularities of the multiple joints 104 of the surgical robot arm 101.

### SUMMARY OF THE INVENTION

According to a first aspect of the invention there is provided a control system for a surgical robotic system, the surgical robotic system comprising a surgical robot arm and a surgeon input device, the surgical robot arm comprising a series of joints by which its configuration can be altered, the series of joints extending from a base at a proximal end of the surgical robot arm to an attachment for a surgical instrument at a distal end of the surgical robot arm, the control system being configured to control the surgical robot arm, in dependence on inputs received at the surgeon input device, to alter the configuration of the surgical robot arm whilst maintaining an intersection between a surgical instrument attached to the surgical robot arm and a fulcrum, in which the control system is configured to: receive an input from the surgeon input device, said input indicating a requested movement of the surgical instrument; determine a scaling factor using a scaling function that is dependent on the distance between a part of the surgical instrument and the fulcrum; generate a control signal for the surgical robot arm in dependence on the input from the surgeon input device and the determined scaling factor; and send the generated control signal to the surgical robot arm in order to drive the surgical robot arm such that its configuration is altered so as to cause movement of the surgical instrument.

The scaling function may output a maximum scaling factor when the distance between the part of the surgical instrument and the fulcrum equals or exceeds an upper threshold.

The maximum scaling factor may be 1.

The scaling function may output a minimum scaling factor when the distance between the part of the surgical instrument and the fulcrum equals or falls below a lower threshold.

The lower threshold may be set in dependence on one or more parameters of the surgical instrument.

The minimum scaling factor may be 0.

The scaling function may be symmetrical between the upper threshold and the lower threshold.

The scaling function may be non-linear.

The scaling function may be a cubic spline.

The cubic spline may be defined by a first end point, an interval point, a second end point, a first derivative at a point between the first end point and the interval point, and a second derivative at a point between the interval point and the second end point.

The interval point may be equidistant between the first end point and the second end point, and the first derivative may be equal to the second derivative.

Said input may indicate a requested translational movement of the part of the surgical instrument.

The surgical instrument may comprise a base at its proximal end by which it attaches to the surgical robot arm and a shaft connecting the base to an articulation, the articulation permitting movement of an end effector at the distal end of the surgical instrument relative to the shaft.

The part of the surgical instrument may be the distal end of the shaft of the surgical instrument.

The control system may be configured to control the surgical robot arm, in dependence on inputs received at the surgeon input device, to alter the configuration of the surgical robot arm whilst maintaining an intersection between the shaft of the surgical instrument and the fulcrum.

The control system may be configured to determine the distance between the part of the surgical instrument and the fulcrum in dependence on the position of the attachment for the surgical instrument and one or more parameters of the surgical instrument.

The one or more parameters may include the length of the shaft.

The fulcrum may be a point in space about which the control system is configured to cause the surgical instrument to pivot.

The control system may be configured to: scale the input from the surgeon input device using the determined scaling factor; and generate the control signal for the surgical robot arm in dependence on the scaled input.

The control system may be configured to generate the control signal for the surgical robot arm further in dependence on a further scaling factor.

The further scaling factor may be determined in dependence on: one or more parameters of the surgical instrument; and/or a received input indicating the surgeon's preference.

According to a second aspect of the invention there is provided surgical robotic system comprising: a surgical robot arm comprising a series of joints by which its configuration can be altered, the series of joints extending from a base at a proximal end of the surgical robot arm to an attachment for a surgical instrument at a distal end of the surgical robot arm; a surgeon input device; and a control system as described herein.

According to a third aspect of the invention there is provided a method of controlling a surgical robotic system, the surgical robotic system comprising a surgical robot arm and a surgeon input device, the surgical robot arm comprising a series of joints by which its configuration can be altered, the series of joints extending from a base at a proximal end of the surgical robot arm to an attachment for a surgical instrument at a distal end of the surgical robot arm, the method comprising controlling the surgical robot arm, in dependence on inputs received at the surgeon input device, to alter the configuration of the surgical robot arm whilst maintaining an intersection between a surgical instrument attached to the surgical robot arm and a fulcrum, in which the method comprises: receiving an input from the surgeon input device, said input indicating a requested movement of the surgical instrument; determining a scaling factor using a scaling function that is dependent on the distance between a part of the surgical instrument and the fulcrum; generating a control signal for the surgical robot arm in dependence on the input from the surgeon input device and the determined scaling factor; and sending the generated control signal to the surgical robot arm in order to drive the surgical robot arm such that its configuration is altered so as to cause movement of the surgical instrument.

According to a fourth aspect of the invention there is provided a non-transitory computer readable storage medium having stored thereon computer readable instructions that, when executed at a control system for a surgical robotic system, cause the control system to perform the method as described herein.

### BRIEF DESCRIPTION OF THE DRAWINGS

The present invention will now be described by way of example with reference to the accompanying drawings. In the drawings:

FIG. 1 shows an example surgical robotic system.

FIG. 2 shows an example surgical instrument.

FIG. 3 shows an example surgical robot arm.

FIG. 4 shows an example surgical robotic system.

FIG. 5 shows an example surgical robot arm calibration process.

FIG. 6 shows a surgical robot arm control process.

FIG. 7 shows an example scaling function.

### DETAILED DESCRIPTION OF THE DRAWINGS

The following description is presented to enable any person skilled in the art to make and use the invention, and is provided in the context of a particular application.

Various modifications to the disclosed examples will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

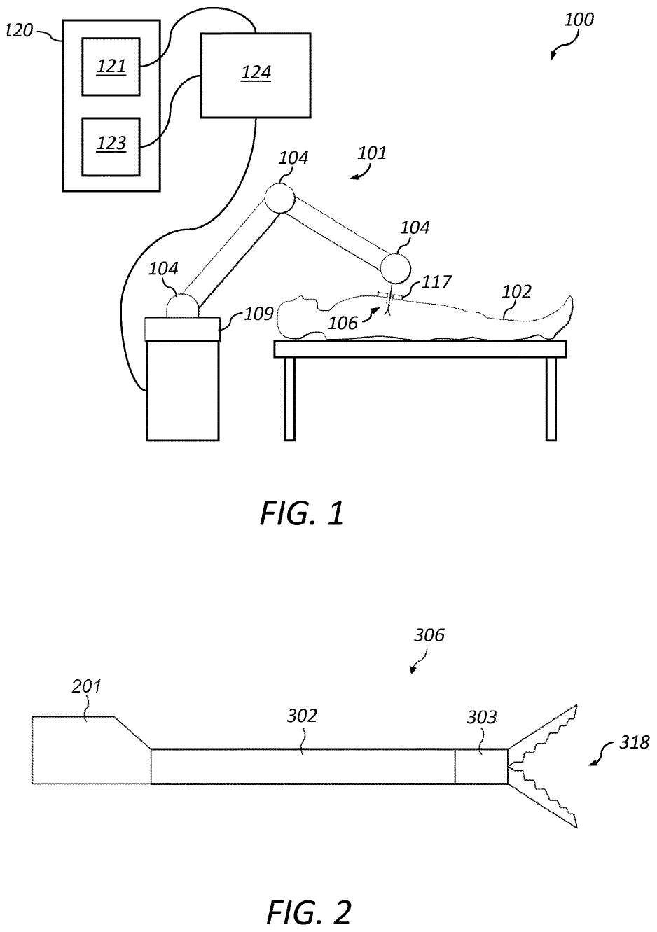

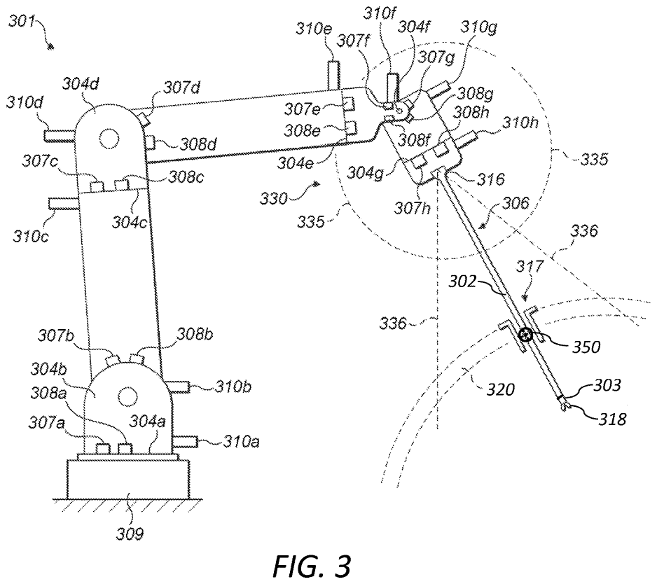

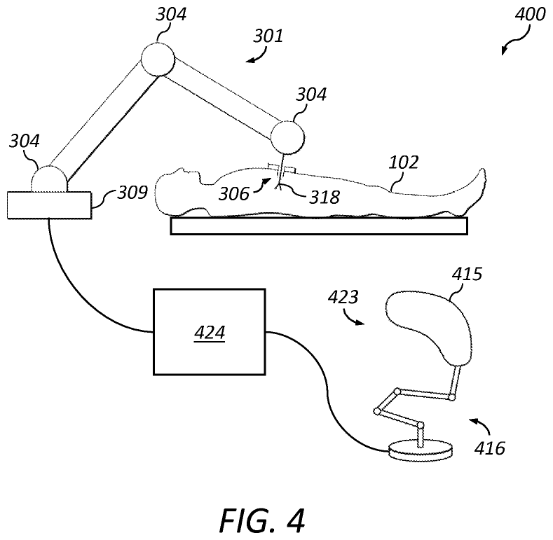

FIG. 3 shows an example of a surgical robot arm 301. The surgical robot arm 301 may be comprised within a surgical robotic system—such as the surgical robotic system shown in FIG. 1, or the surgical robotic system shown in FIG. 4 as will be described in further detail herein.

At its proximal end, the surgical robot arm 301 comprises a base 309. The surgical robot arm has a series of rigid arm members. Each arm member in the series is joined to the preceding arm member by a respective joint 304—shown in FIG. 3 as joints 304a-g. Joints 304a-g may be referred to as a series of joints. Joints 304a-e and 304g are revolute joints. Joint 304f is composed of two revolute joints whose axes are orthogonal to each other, e.g. as in a “Hooke's” or universal joint. Joint 304f may be termed a “wrist joint”. A surgical robot arm could be jointed differently from the surgical robot arm 301 of FIG. 3. For example, joint 304d could be omitted and/or joint 304f could permit rotation about a single axis. Alternatively, or additionally, the surgical robot arm 301 could include one or more joints that permit motion other than rotation between respective sides of the joint, such as a prismatic joint by which an instrument attachment can slide linearly with respect to more proximal parts of the surgical robot arm.

The joints are configured such that the configuration of the surgical robot arm can be altered. This allows the distal end 330 of the surgical robot arm to be moved to an arbitrary point in a three-dimensional working volume illustrated generally at 335. One way to achieve that is for the joints to have the arrangement illustrated in FIG. 3. Other combinations and configurations of joints could achieve a similar range of motion. There could be more or fewer arm members.

The distal end 330 of the surgical robot arm 301 has an attachment 316 by means of which a surgical instrument 306 can be releasably attached. Movement of the surgical robot arm 301 thereby causes movement of the surgical instrument 306. The surgical instrument 306 has a shaft 302. The surgical instrument 306 has an end effector 318 at the distal end of the shaft 302. The end effector 318 consists of a device for engaging in a procedure, for example a cutting, grasping or imaging device. As described herein, terminal joint 304g may be a revolute joint. The surgical instrument 306 and/or the attachment 316 may be configured so that the surgical instrument 306 (e.g. in particular, its shaft 302) extends linearly parallel with the rotation axis of the terminal joint 304g of the surgical robot arm 301. In this example the surgical instrument 306 (e.g. in particular, its shaft 302) extends along an axis coincident with the rotation axis of joint 304g.

For some types of minimally invasive procedure, the surgical instrument 306 may access a surgical site within the patient's body through a synthetic access port 317. For example, the minimally invasive procedure may be performed at a surgical site within the patient's abdomen. The port 317 may provide a passageway through the outer tissues 320 of the patient so as to limit disruption to those tissues as the surgical instrument 306 is inserted and removed, and as the surgical instrument 306 is moved by the surgical robot arm 301 about the surgical site. For other types of minimally invasive procedure, the surgical instrument 306 may access a surgical site within the patient's body directly through a natural orifice. For example, the minimally invasive procedure may be performed at a surgical site in the patient's throat, and the natural orifice may be the patient's mouth.

FIG. 2 shows in more detail an example surgical instrument for attachment to the surgical robot arm 301 shown in FIG. 3. The surgical instrument 306 comprises a base 201 at its proximal end by which it connects to (e.g. attaches to) the surgical robot arm 301. A shaft 302 connects the base 201 to an articulation 303. The shaft 302 is a rigid linear shaft. The articulation 303 is connected to the distal end of the shaft 302. The articulation 303 connects the shaft 302 to an end effector 318. The end effector 318 is at the distal end of the surgical instrument 306. By way of example only, in FIG. 2, a pair of serrated jaws are illustrated as the end effector 318. The articulation 303 permits the end effector 318 to move relative to the shaft 302. The skilled person would be aware of numerous articulations suitable for permitting the end effector 318 to move relative to the shaft 302, and so for conciseness the specific implementation of the articulation will not be discussed further herein.

It is to be understood that FIG. 2 shows just one specific example of a surgical instrument, and that various other suitable surgical instruments exist to which the principles described herein could be applied.

Returning to FIG. 3, joints 304e and 304f of the surgical robot arm 301 are configured so that, with the distal end 330 of the surgical robot arm 301 held at an arbitrary location in the working volume 335, the surgical instrument 306 can be moved in an arbitrary direction within a cone 336. One way to achieve that is for the terminal part of the arm to comprise the pair of joints 304e and 304f whose axes are mutually arranged as described above. Other mechanisms can achieve a similar result. For example, joint 304g could influence the attitude of the instrument if the instrument extends in a direction which is not parallel to the axis of joint 304g.

The surgical robot arm 301 comprises a series of motors 310a-h. With the exception of the compound joint 304f, which is served by two motors, each motor is arranged to drive rotation about a respective joint of the surgical robot arm 301. The motors are controlled by a control system (such as control system 124 shown in FIG. 1, or the control system 424 shown in FIG. 4 as will be described in further detail herein). The control system comprises a processor and a memory. The memory stores, in a non-transient way, software code that can be executed by the processor to cause the processor to control the motors 310a-h in order to alter the configuration of the surgical robot arm 301 in the manner described herein.

The surgical robot arm 301 may comprise a series of sensors 307a-h and 308a-h. These sensors may comprise, for each joint, a position sensor 307a-h for sensing the rotational position of the joint and a force sensor 308a-h for sensing forces (or torques) applied about the joint's rotation axis. Compound joint 304f may have two pairs of sensors. One or both of the position and force sensors for a joint may be integrated with the motor for that joint. The outputs of the sensors are passed to the control system (such as control system 124 shown in FIG. 1, or the control system 424 shown in FIG. 4 as will be described in further detail herein) where they form inputs for the processor.

It is to be understood that FIG. 3 shows just one specific example of a surgical robot arm, and that various other suitable surgical robot arms exist to which the principles described herein could be applied.

FIG. 4 shows an example a surgical robotic system 400 comprising a surgical robot arm 301 and a surgeon input device 423.

A simplified schematic of the surgical robot arm 301 is shown in FIG. 4 for ease of illustration. It is to be understood that the surgical robot arm 301 shown in FIG. 4 can have the same properties and features as the surgical robot arm 301 described with reference to FIG. 3.

In FIG. 4, the surgeon input device 423 comprises a hand controller 415 connected to a gimbal assembly 416 which permits the hand controller 415 to move, e.g. with six degrees of freedom. A simplified schematic of the gimbal assembly is shown in FIG. 4 for ease of illustration. The configuration of the gimbal assembly 416 can be detected by sensors on the gimbal assembly 416 and passed to the control system 424. A surgeon can move the hand controller 315 in order to request corresponding movement of the surgical instrument 306 attached to the surgical robot arm 301. The skilled person would be aware of numerous gimbal assemblies suitable for permitting the hand controller 315 to move with six degrees of freedom and for detecting that movement, and so for conciseness the specific implementation of the gimbal assembly will not be discussed further herein. In an alternative example, instead of the gimbal assembly 416, the hand controller 415 could be equipped with accelerometers which permit its position and orientation to be estimated.

It is to be understood that FIG. 4 shows just one specific example of a surgeon input device, and that various other suitable surgeon input devices exist to which the principles described herein could be applied. For example, the surgeon input device may instead resemble a “games console controller” having a plurality of joysticks that can be moved to request corresponding movement of the surgical robot arm 301.

The surgeon input device 423 may be comprised by a surgeon console that is remote from the surgical robot arm 301. Other components of the remote surgeon console, such as a display, are not shown in FIG. 4 for ease of illustration.

A control system 424 connects the surgeon input device 423 to the surgical robot arm 301. The control system 424 may be separate from the remote surgeon console and the surgical robot arm 301. The control system 424 may be collocated with the remote surgeon console. The control system 424 may be collocated with the surgical robot arm 301. The control system 424 may be distributed between the remote surgeon console and the surgical robot arm 301.

The control system 424 comprises a processor and a memory. The memory stores, in a non-transient way, software code that can be executed by the processor to cause the processor to control the surgical robot arm 301 in the manner described herein.

The control system 424 receives inputs from the surgeon input device 423 and converts those inputs to control signals to move one or more of the joints 304 of the surgical robot arm 301 in order to alter its configuration. The control signals can be generated based on kinematic equations that define the relationship between the position of the one or more joints 304 and the position of the attachment 316 for the surgical instrument—as would be well understood by the skilled person. The control system 424 sends these control signals to the surgical robot arm 301, where the corresponding one or more of the joints 304 are driven accordingly. Movement of the surgical instrument 306 attached to the surgical robot arm 301 can thereby be controlled by the control system 424 in response to movement of the surgeon input device 423.

The control system 424 can use a control relationship to transform an input from the surgeon input device 423 so as to generate a control signal for causing movement of the surgical instrument 306. A position control relationship is an example type of control relationship in which the position of a surgeon input device 423 dictates the position of the surgical instrument 306 (e.g. in particular, a part of the surgical instrument 306 such as the distal end of its shaft 302 or its end effector 318). For example, referring to FIG. 4, using a position control relationship, the position of the hand controller 415 in the hand controller workspace can be directly converted to a position of the end effector 318 in the end effector workspace. More specifically, the control system 424 may determine the position of the hand controller 415 at a plurality of instances in time. The position of the hand controller 415 can be determined using the inputs from the sensors on the gimbal assembly 416. The position of the hand controller 415 can be determined irregularly or at predetermined time intervals, e.g. every 0.4 milliseconds (ms). In other words, the position of the hand controller 415 can be determined at a predetermined frequency, e.g. of 2.5 kHz The difference in the position of the hand controller between two instances in time can be determined. For example, it may be determined that in the preceding 0.4 ms interval the position of the hand controller 415 changed by 0.1 millimetres (mm) in a certain direction. In response, using a position control relationship, the control system 424 may generate a control signal in order to drive the surgical robot arm 301 such that its configuration is altered so as to cause the position of the end effector of the surgical instrument to also change by 0.1 mm in that certain direction. It is to be understood that the control system 424 need not necessarily generate control signals for causing the end effector to change position at the same frequency as the frequency at which the position of the hand controller 415 is determined. For example, the position of the hand controller 415 may be determined at a frequency of 5 kHz whilst control signals for the surgical robot arm 301 are generated at a frequency of 2.5 kHz. In this example, each generated control signal may cause the position of the end effector to change by the distance and in the direction that the hand controller has moved in the preceding two time intervals, e.g. two 0.2 ms time intervals. It is also to be understood that, as will be described in further detail herein, the relationship between the distance moved by the hand controller and the resultant distance moved by end effector need not be 1:1 (e.g. 0.1 mm:0.1 mm) as it is in the example above.

Constraints may be placed on the movement of the surgical instrument that can be caused by the control system. One such constraint is that the control system 424 is configured to control the surgical robot arm 301, in dependence on inputs received at the surgeon input device 423, to alter the configuration of the surgical robot arm 301 whilst maintaining an intersection between the surgical instrument 306 attached to the surgical robot arm 301 and a fulcrum. The control system may be configured to control the surgical robot arm 301 in this way during a minimally invasive procedure. FIG. 3 shows an example fulcrum 350. The fulcrum 350 is a point in space about which the control system 424 is configured to cause the surgical instrument 306 to pivot. The fulcrum may be referred to as a “pivot point” or a “virtual pivot point”. The fulcrum 350 may be mechanically enforced or may be a software constraint enforced by the control system 424. In the example shown in FIG. 3, there is nothing physically present at the fulcrum (which is why it may be referred to as a virtual pivot point or a virtual fulcrum) and the fulcrum 350 is a software constraint enforced by the control system 424 when it determines the control signals for driving the surgical robot arm 301.

As such, the surgical robot arm 301 is driven using control signals which maintain an intersection between the surgical instrument 306 and the pivot point 350. For example, during the minimally invasive procedure, the surgeon can use the surgeon input device 423 to indicate a desired position of the surgical instrument 306 (e.g. in particular, a part of the surgical instrument 306 such as its end effector 318). In response, the control system 424 determines a configuration of the series of joints 304 of the surgical robot arm 301 that will result in both (i) the end effector 318 of the surgical instrument 306 being placed in that desired position and (ii) the shaft 302 of the surgical instrument 306 passing through (e.g. maintaining an intersection with) the fulcrum 350, and to generate a control signal to move the series of joints 304 to that configuration.

By determining a suitable fulcrum, the disruption to the outer tissues of the patient caused by moving the surgical instrument 306 during a minimally invasive procedure can be minimised. For example, a suitable fulcrum may be located within the port 317, e.g. at or close to the centre of the port 317.

In a simple example, a user of the surgical robotic system 400 may determine the fulcrum “by eye”. For example, prior to a minimally invasive procedure, the distal end of the shaft 302 of the surgical instrument 306 may be positioned by the user within the access port or natural orifice. When the user is satisfied that the distal end of the shaft 302 is positioned in the centre of the access port or natural orifice, they can signal to the control system 424 that the current position of the distal end of the shaft 302 should be saved as the fulcrum. The control system may determine the current position of the distal end of the shaft 302 in dependence on inputs from the position sensors 307a-h that indicate the position of the attachment 316 for the surgical instrument 306 and one or more parameters of the surgical instrument 306 (e.g. including the length of its shaft, and the orientation of its shaft relative to its base) stored in the memory of the control system or stored in a memory on the instrument itself. The control system can then store this fulcrum in memory for later use.

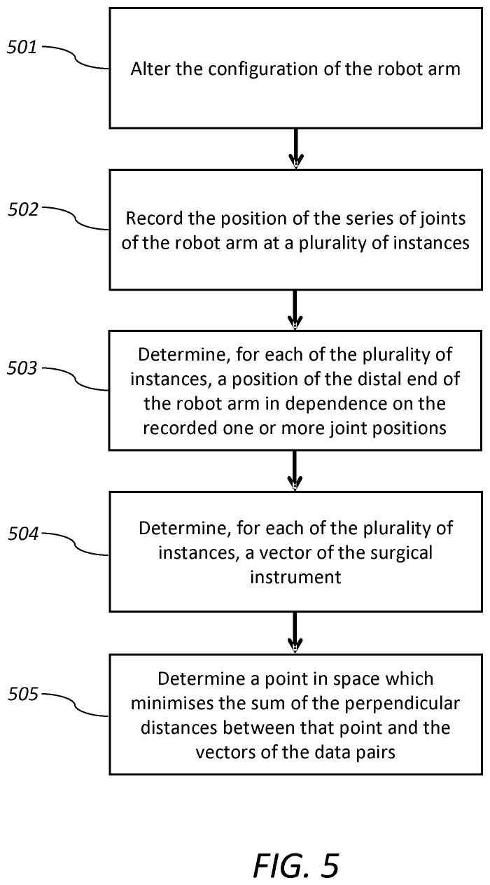

In an alternative example, a calibration process can be performed prior to performing a minimally invasive procedure in order to determine a suitable fulcrum. FIG. 5 shows an example surgical robot arm calibration process. The operating mode of the surgical robotic system 400 can be set to be a calibration mode during the calibration process so that the surgical robotic system can act accordingly in order to calibrate the surgical robot.

In step 501, the configuration of the surgical robot arm 301 can be altered 501 whilst the surgical instrument 306 is inside the access port 317 or natural orifice.

During the calibration process, the configuration of the surgical robot arm 301 can be altered by the application of external forces directly onto the surgical robot arm 301. For example, the user (e.g. a surgeon or a member of the operating room staff) may apply forces directly to the surgical robot arm 301 (e.g. by pushing a joint of the surgical robot arm 301)—which can be sensed by the force sensors 308a-h and acted on by the control system 424 in a manner that would be understood by the skilled person. During the calibration process, when operating in the calibration mode, the control system 124 can control the surgical robot arm 301 to maintain a position in which it is placed by means of external forces applied directly to the surgical robot arm 301.

During the calibration process, when operating in the calibration mode, the surgical robot arm 301 can be moved generally transversely to the shaft 302 of the surgical instrument 306. The configuration of the surgical robot arm 301 may be altered such that the distal end of the surgical robot arm 301 is moved in two dimensions transverse (e.g. perpendicular) to the shaft 302: e.g. with (i) components parallel to a direction that is transverse to the shaft 302 and also with (ii) components orthogonal to that direction but transverse to the shaft 302. To do this, the user (e.g. a surgeon or a member of the operating room staff) may gyrate the distal end 330 of the surgical robot arm 301 about a point generally aligned with the natural axis of the access port or natural orifice. This causes the surgical instrument 306 to come into contact with the access port 317 (or natural orifice) such that the access port 317 (or natural orifice) applies a lateral force on the shaft 302. That force can be accommodated by motion about the joint 204f. The force is “lateral” in the sense that it is applied to the sides of the instrument and is generally in a direction that is transverse (e.g. perpendicular) to the shaft 302 of the instrument 306.

In step 502, as the configuration of the surgical robot arm 301 is being altered, the position sensors 307a-h can record the position of each joint 304 of the series of joints of the surgical robot arm 301. The position sensors 307a-h can record the positions of each joint of the surgical robot arm 301 at a plurality of instances in time. Position information may be recorded irregularly or at predetermined time intervals, e.g. every 20 milliseconds (ms). In other words, position information may be recorded at a predetermined frequency, e.g. of 50 Hz. The position sensors provide the recorded position information to the control system 424. The control system may also store in memory information indicating one or more parameters of the surgical instrument 306 (e.g. including the length of the shaft and/or the orientation of its shaft relative to its base). These parameters of the surgical instrument 306 may be read from a memory on the surgical instrument itself and passed to the control system 424.

In steps 503 and 504, the control system 424 uses this information to determine, at each of the plurality of instances in time: (a) the position of the distal end 330 of the surgical robot arm 301 relative to the base 309 and (b) the vector representing the surgical instrument 306 (e.g. in particular, its shaft 302) relative to the distal end 330 of the surgical robot arm 301. Position (a) and vector (b) may be termed a data pair.

The vectors of the data pairs will approximately (but usually not exactly) converge, from their respective distal end position, on the natural rotation centre of the access port 317 or natural orifice. By collecting a plurality of said data pairs, and then solving for a best estimate (i.e. an estimate with the least error) of a location where the vectors converge, the control system 424 can determine a fulcrum (e.g. a pivot point) within the access port or natural orifice. For example, in step 505, the control system 424 may estimate, as the fulcrum, the point in space which minimises the sum of the perpendicular distances between that point and the vectors of the data pairs. Here, a “perpendicular distance” between the point and a vector refers to the distance between the point and the vector in a direction perpendicular to the vector. Therefore, the perpendicular distance between the point and a vector is the distance between the point and the position on the vector which is closest to the point. In some examples, the control system 424 may estimate, as the fulcrum, the point in space which minimises the sum of the squares of the perpendicular distances between that point and the vectors of the data pairs. The control system 424 can store this fulcrum in memory for later use.

During the minimally invasive procedure, it is desirable for the control system 424 to control the surgical robot arm 301 such that the surgical instrument 306 can access as much of the surgical site as possible. That said, it has been observed that problems can be experienced when the surgical instrument 306 (in particular, the distal end of its shaft) moves close to the fulcrum (e.g. within 5 centimetres (cm) of the fulcrum).

One such problem is that, when the distal end of the shaft is particularly close to the fulcrum, parts of the surgical instrument (e.g. more proximal parts of its shaft, or its end effector) can begin to unintentionally impinge on the access port or the natural orifice—and thereby cause disruption to the outer tissues of the patient—as a result of the control system causing movements of the surgical instrument whilst the surgical instrument pivots about the nearby fulcrum.

Another problem is that, when the distal end of the shaft is particularly close to the fulcrum, the surgical robot arm 301 approaches a kinematic singularity. This means that extremely large movements of one or more of the joints 304 of the surgical robot arm 301 can be required to cause relatively small lateral movements of the distal end of the surgical instrument 306. Extremely large movements of the surgical robot arm 301 such as these can be undesirable, as they can cause injuries to unsuspecting members of the operating room staff, or cause collisions between multiple surgical robot arms comprised by the same surgical robotic system.

One solution to these problems is for the control system 424 to be configured to prevent a part of the surgical instrument 306 (e.g. in particular, the distal end of its shaft) from being positioned less than a predetermined distance (e.g. less than 5 cm) from the fulcrum. That is, the control system 424 could enforce a “keep-out zone”—a volume having a predetermined radius (e.g. 5 cm) around the fulcrum that the distal end of the shaft is prevented from entering. In other words, movement of the distal end of the shaft towards the fulcrum may be abruptly halted when the control system 424 determines that the distance between the distal end of the shaft and the fulcrum equals the predetermined distance (e.g. 5 cm).

That said, it has been observed that this solution is not optimal. This is because, for certain types of minimally invasive procedure (e.g. such as hernia operations) it can be desirable to access portions of the surgical site which often fall within that predetermined distance (e.g. 5 cm) from the fulcrum. As such, this solution could be perceived to be overly restrictive by surgeons using the surgical robotic system 400.

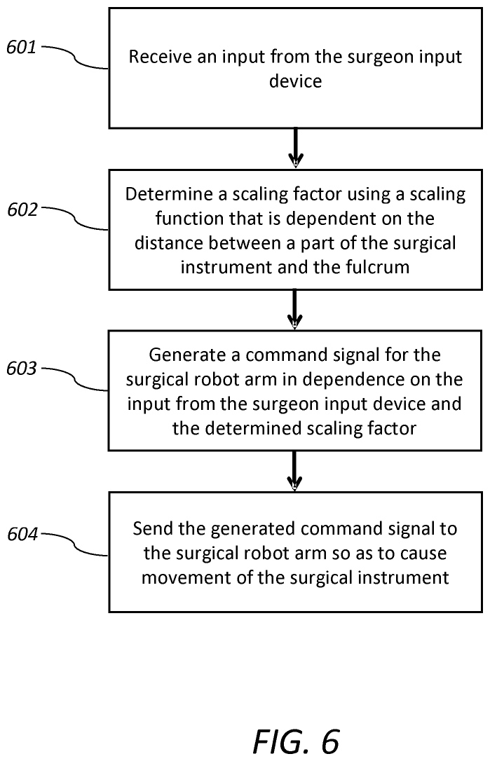

FIG. 6 shows a surgical robot arm control process. The control system 424 of the surgical robotic system 400 shown in FIG. 4 can be configured to perform this control process so as to address one or more of the problems described above.

In step 601, the control system 424 is configured to receive an input from the surgeon input device 423, said input indicating a requested movement of the surgical instrument 306. As described herein with reference to FIG. 4, the surgeon input device 423 may comprise a hand controller 415 that the surgeon can move in order to request corresponding movement of the surgical instrument 306 attached to the surgical robot arm 301.

The input may indicate a request for a translational movement (e.g. a position change) of a part of the surgical instrument 306 (e.g. the distal end of its shaft 302 or its end effector 318). For example, from the input received from the surgeon input device 423 the control system 424 may determine the position of the hand controller 415 at a plurality of instances in time. The position of the hand controller 415 can be determined using the inputs from the sensors on the gimbal assembly 416. The position of the hand controller 415 can be determined irregularly or at predetermined time intervals, e.g. every 0.4 milliseconds (ms). In other words, the position of the hand controller 415 can be determined at a predetermined frequency, e.g. of 2.5 kHz. The difference in the position of the hand controller between two instances in time can be determined. For example, it may be determined that in the preceding 0.4 ms interval the position of the hand controller 415 changed by 0.1 mm in a certain direction. This may be interpreted by the control system 424 as a request to move the part of the surgical instrument 306 by 0.1 mm in that certain direction. It is to be understood that the input may indicate a request to move the part of the surgical instrument by the distance and in the direction moved by the hand controller 415 in more than one preceding time interval (e.g. in examples where the frequency at which the position of the hand controller is measured is different to the frequency at which control signals for the surgical robot arm 301 are generated).

It is to be understood that the principles described herein could also be applied to an input indicating a requested rotational movement (e.g. an orientation change) of a part of the surgical instrument.

In step 602, the control system 424 is configured to determine a scaling factor using a scaling function that is dependent on the distance between a part of the surgical instrument and the fulcrum.

The control system 424 may be configured to determine the distance between the part of the surgical instrument 306 (e.g. the distal end of its shaft 302 or its end effector 318) and the fulcrum 350 in dependence on the position of the attachment 316 for the surgical instrument and one or more parameters of the surgical instrument 306. For example, the control system may determine the position of the distal end of the shaft 302 in dependence on: inputs from the position sensors 307a-h that indicate the position of the attachment 316 for the surgical instrument 306; and parameters of the surgical instrument 306 indicating the length of its shaft, and the orientation of its shaft relative to its base, that are stored in the memory of the control system 424. As described herein, the position of the fulcrum 350 is also stored in the memory of the control system 424. As such, the control system 424 can determine the distance between the distal end of the shaft 302 of the surgical instrument 306 and the fulcrum 350 by assessing the difference in their positions.

The scaling function may be stored in the memory of the control system 424. The control system 424 can be configured to use the scaling function to map the determined distance between the part of the surgical instrument 306 and the fulcrum 350 to a scaling factor.

The scaling function may output a maximum scaling factor when the distance between the part of the surgical instrument and the fulcrum equals or exceeds an upper threshold. The maximum scaling factor may be 1. By way of example only, the upper threshold may be 6 cm.

The scaling function may output a minimum scaling factor when the distance between the part of the surgical instrument and the fulcrum equals or falls below a lower threshold. The minimum scaling factor may be 0. By way of example only, the lower threshold may be 1 cm. The lower threshold may be set in dependence on one or more parameters of the surgical instrument 306. For example, the lower threshold may be set to be greater than or equal to the length of the articulation 303 of the surgical instrument 306.

The scaling factor output by the scaling function may decrease (e.g. gradually decrease) between the upper threshold and the lower threshold.

The scaling function may be linear. For example, the scaling factor output by the scaling function may decrease linearly (e.g. from 1 to 0) as the distance between the part of the surgical instrument 306 and the fulcrum 350 decreases from the upper threshold to the lower threshold. The scaling function may comprise two or more linear sections of different gradients. For example, the scaling factor output by the scaling function may decrease linearly relatively slowly (e.g. from 1 to 0.75) as the distance between the part of the surgical instrument 306 and the fulcrum 350 decreases from the upper threshold to halfway between the upper and lower thresholds, the scaling factor output by the scaling function may then decrease linearly relatively quickly (e.g. from 0.75 to 0) as the distance between the part of the surgical instrument 306 and the fulcrum 350 decreases from halfway between the upper and lower thresholds to the lower threshold. The scaling function may be non-linear. For example, the scaling function may be a 1/x function, or may approximate a 1/x function. The scaling function may comprise two or more non-linear sections of different curvature. The scaling function may comprise a mixture of one or more linear sections and one or more non-linear sections.

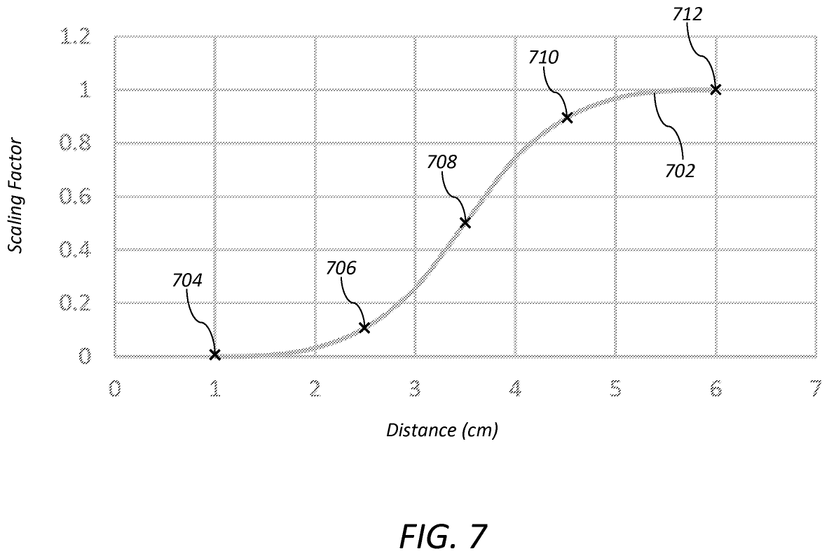

FIG. 7 shows an example scaling function 702. The scaling function 702 has been observed to be particularly advantageous. The scaling function 702 is a cubic spline. The cubic spline is defined by a first end point 704, an interval point 708, a second end point 712, a first derivative at a point 706 between the first end point 704 and the interval point 708, and a second derivative at a point 710 between the interval point 708 and the second end point 712. The first end point 704 is at the lower threshold (e.g. 1 cm in this example). The second end point 712 is at the upper threshold (e.g. 6 cm in this example). In this illustrative example, first end point 704 is at (1, 0), the interval point 708 is at (3.5, 0.5) and the second end point 712 is at (6, 1).

In scaling function 702, the interval point 708 is equidistant between the first end point 704 and the second end point 712, and the first derivative is equal to the second derivative. This means that the scaling function 702 is symmetrical between the upper threshold and the lower threshold. This need not be the case. Alternatively, the scaling function 702 may be non-symmetrical between the upper threshold and the lower threshold—e.g. the interval point 708 may not be equidistant between the first end point 704 and the second end point 712, and/or the first derivative may not be equal to the second derivative.

To give an illustrative example, the control system 424 may use the scaling function 702 to map a determined distance of 3.5 cm between the part of the surgical instrument 306 and the fulcrum 350 to a scaling factor of 0.5.

It is to be understood that FIG. 7 shows just one specific example of a scaling function, and that various other suitable scaling functions could be used to achieve the advantages described herein. In particular, a different scaling function could be used for each different type of surgical instrument that can be attached to the surgical robot arm. That is, a set of scaling functions may be stored in the memory of the control system 424, whereby the control system 424 selects one of those scaling functions for use in the control process of FIG. 6 in dependence on the type of surgical instrument attached to the surgical robot arm 301.

In step 603, the control system 424 is configured to generate a control signal for the surgical robot arm in dependence on the input from the surgeon input device 423 and the determined scaling factor.

For example, the control system 424 may be configured to scale the input from the surgeon input device using the determined scaling factor. Said scaling may comprise multiplying the movement requested by the input from the surgeon input device 423 by the determined scaling factor.

Building on the illustrative examples set out above, the input from the surgeon input device 423 may be indicative of a request to move the part of the surgical instrument 306 by 0.1 millimetres (mm) in a certain direction, and the distance between the part of the surgical instrument 306 and the fulcrum 350 may be 3.5 centimetres (cm) such that the control system determines a scaling factor of 0.5. In this example, the input can be scaled such that, conceptually, the indicated requested movement of 0.1 mm in a certain direction is multiplied by the determined scaling factor of 0.5 so as to output a scaled input indicating a requested movement of 0.05 mm in that certain direction.

The control system 424 may be configured to generate the control signal for the surgical robot arm 301 in dependence on the scaled input. That is, the control system 424 may be configured to generate a control signal for driving the surgical robot arm such that its configuration is altered so as to cause movement of the surgical instrument in accordance with (e.g. so as to comply with the requested movement indicated by) the scaled input.

It is also to be understood that the control system may be configured to generate the control signal for the surgical robot arm further in dependence on a further scaling factor. That is, the control signal for the surgical robot arm may be generated in step 603 in dependence on one or more further scaling factors in addition to the scaling factor determined in step 602. For example, a further scaling factor can be determined in dependence on one or more parameters of the surgical instrument—e.g. a relatively lower (i.e. closer to 0) further scaling factor can be determined for surgical instruments having shafts of relatively greater length. In another example, a further scaling factor can be determined in dependence on a received input indicating the surgeon's preference. By way of example only, the surgeon may request a further scaling factor of 0.5 be applied to all inputs received from the surgeon input device regardless of the distance of between the part of the surgical instrument 306 and the fulcrum 350—e.g., such that a movement of the hand controller 415 of 0.1 mm in a direction is scaled by the further scaling factor to a requested movement of the surgical instrument of 0.05 mm in that direction. In these examples, the control system 424 may be configured to scale the input from the surgeon input device using each of the one or more further scaling factors in addition to the scaling factor determined in step 602—e.g. by multiplying the movement requested by the input from the surgeon input device 423 by each of the one or more further scaling factors and the scaling factor determined in step 602.

In step 604, the control system 424 is configured to send the generated control signal to the surgical robot arm 301 in order to drive the surgical robot arm 301 such that its configuration is altered so as to cause movement of the surgical instrument 306. Step 604 can be understood with reference the description of FIGS. 3 and 4 herein.

As described herein, the control system 424 may be configured to generate a control signal for the surgical robot arm at predetermined time intervals, e.g. every 0.4 milliseconds (ms). In other words, the control system 424 may be configured to generate control signals for the surgical robot arm at a predetermined frequency, e.g. of 2.5 kHz. As such, steps 601, 602, 603 and 604 can be repeated at each predetermined time interval or at the predetermined frequency, e.g. every 0.4 ms or with a frequency of 2.5 kHz, so as to control the movement of the surgical instrument 306.

The surgical robot arm control process described herein with reference to FIG. 6 is advantageous as it enables the surgical instrument to access more of the surgical site than previous approaches. Owing to the use of a scaling function as described herein, the movement of the surgical instrument can be gradually slowed as the distance between the part of the surgical instrument and the fulcrum decreases. This is because, for the same input from the surgeon input device, the resultant change in surgical instrument position per unit time would be less when the surgical instrument is closer to the fulcrum than when it is further from the fulcrum. This means that the speed with which the surgical robot arm is required to alter its configuration so as to cause movement of the surgical instrument is also gradually reduced as the surgical instrument approaches the fulcrum. This reduces the likelihood of injury to members of the operating room staff or damage to other equipment in the operating room, even when the movements of the joints 304 are large as the surgical robot arm 301 approaches a kinematic singularity. Furthermore, the forces that could be exerted by parts of the surgical instrument were they to unintentionally impinge on the access port or the natural orifice are also reduced when the movement of surgical instrument is gradually slowed as it approaches the fulcrum. It is also preferable for the surgeon that the movement of the surgical instrument is slowed gradually in the control process described herein, rather than abruptly as in previous approaches.

It is to be understood that the lower threshold of the scaling function can act to define a “keep-out zone”—a volume having a predetermined radius (e.g. 1 cm, for a lower threshold of 1 cm) around the fulcrum that the part of the instrument is prevented from entering. That said, owing to the scaling of the inputs between the upper threshold and the lower threshold as described herein, this “keep-out zone” can advantageously be significantly smaller than in previous approaches.

It is to be understood that the robot arm described herein could be for purposes other than surgery. For example, the access port could be an inspection port in a manufactured article such as a car engine and the robot arm could control a viewing instrument for viewing inside the engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system configured to control a surgical robot arm that is comprised by a surgical robotic system, the surgical robotic system comprising the surgical robot arm and a surgeon input device, the surgical robot arm comprising a series of joints by which its configuration can be altered, the series of joints extending from a base at a proximal end of the surgical robot arm to an attachment configured to receive a surgical instrument at a distal end of the surgical robot arm, the control system being configured to control the surgical robot arm, in dependence on inputs received at the surgeon input device, to alter the configuration of the surgical robot arm whilst maintaining an intersection between a surgical instrument attached to the surgical robot arm and a fulcrum, in which the control system is configured to:
receive an input from the surgeon input device, said input indicating a requested movement of the surgical instrument;
determine a scaling factor using a scaling function that is dependent on the distance between a part of the surgical instrument and the fulcrum;
generate a control signal to control the surgical robot arm in dependence on the input from the surgeon input device and the determined scaling factor; and
send the generated control signal to the surgical robot arm in order to drive the surgical robot arm such that its configuration is altered so as to cause movement of the surgical instrument.

2. The control system of claim 1, wherein the scaling function outputs a maximum scaling factor when the distance between the part of the surgical instrument and the fulcrum equals or exceeds an upper threshold.

3. The control system of claim 2, wherein the maximum scaling factor is 1.

4. The control system of claim 1, wherein the scaling function outputs a minimum scaling factor when the distance between the part of the surgical instrument and the fulcrum equals or falls below a lower threshold.

5. The control system of claim 4, wherein the lower threshold is set in dependence on one or more parameters of the surgical instrument.

6. The control system of claim 5, wherein the minimum scaling factor is 0.

7. (canceled)

8. The control system of claim 1, wherein the scaling function is non-linear.

9. The control system of claim 1, wherein the scaling function is a cubic spline.

10. The control system of claim 9, wherein the cubic spline is defined by a first end point, an interval point, a second end point, a first derivative at a point between the first end point and the interval point, and a second derivative at a point between the interval point and the second end point.

11. The control system of claim 9, wherein the interval point is equidistant between the first end point and the second end point, and the first derivative is equal to the second derivative.

12. (canceled)

13. The control system of claim 1, said input indicating a requested translational movement of the part of the surgical instrument.

14. The control system of claim 1, wherein the surgical instrument comprises a base at its proximal end by which it attaches to the surgical robot arm and a shaft connecting the base to an articulation, the articulation permitting movement of an end effector at the distal end of the surgical instrument relative to the shaft.

15. The control system of claim 14, wherein the part of the surgical instrument is the distal end of the shaft of the surgical instrument.

16. (canceled)

17. The control system of claim 1, the control system being configured to determine the distance between the part of the surgical instrument and the fulcrum in dependence on the position of the attachment and one or more parameters of the surgical instrument.

18. (canceled)

19. The control system of claim 1, wherein the fulcrum is a point in space about which the control system is configured to cause the surgical instrument to pivot.

20. The control system of claim 1, the control system being configured to:
scale the input from the surgeon input device using the determined scaling factor; and
generate the control signal to control the surgical robot arm in dependence on the scaled input.

21. The control system of claim 1, the control system being configured to generate the control signal to control the surgical robot arm further in dependence on a further scaling factor.

22. The control system as claimed in claim 21, wherein the further scaling factor is determined in dependence on:
one or more parameters of the surgical instrument; and/or
a received input indicating the surgeon's preference.

23. (canceled)

24. A method of controlling a surgical robotic system, the surgical robotic system comprising a surgical robot arm and a surgeon input device, the surgical robot arm comprising a series of joints by which its configuration can be altered, the series of joints extending from a base at a proximal end of the surgical robot arm to an attachment configured to receive a surgical instrument at a distal end of the surgical robot arm, the method comprising controlling the surgical robot arm, in dependence on inputs received at the surgeon input device, to alter the configuration of the surgical robot arm whilst maintaining an intersection between a surgical instrument attached to the surgical robot arm and a fulcrum, in which the method comprises:
receiving an input from the surgeon input device, said input indicating a requested movement of the surgical instrument;
determining a scaling factor using a scaling function that is dependent on the distance between a part of the surgical instrument and the fulcrum;
generating a control signal to control the surgical robot arm in dependence on the input from the surgeon input device and the determined scaling factor; and
sending the generated control signal to the surgical robot arm in order to drive the surgical robot arm such that its configuration is altered so as to cause movement of the surgical instrument.

25. A non-transitory computer readable storage medium having stored thereon computer readable instructions that, when executed at a control system configured to control a surgical robotic system, cause the control system to perform the method of claim 24.
